(19) United States
(12) Patent Application Publication
(10) Pub. No.: US 2026/0157712 A1
(43) Pub. Date: Jun. 11, 2026

(54) INCREASED CONE BEAM COMPUTED TOMOGRAPHY VOLUME LENGTH WITHOUT REQUIRING STITCHING OR LONGITUDINAL C-ARM MOVEMENT

(71) Applicant: GLOBUS MEDICAL, INC., AUDUBON, PA (US)

(72) Inventors: Neil R. Crawford, Chandler, AZ (US); Norbert Johnson, North Andover, MA (US)

(21) Appl. No.: 19/179,395

(22) Filed: Apr. 15, 2025

Related U.S. Application Data
(63) Continuation of application No. 18/393,847, filed on Dec. 22, 2023, now Pat. No. 12,295,765.
Application No. 18/393,847 is a continuation of application No. 18/055,509, filed on Nov. 15, 2022, now Pat. No. 11,890,122.
Application No. 18/055,509 is a continuation of application No. 17/030,581, filed on Sep. 24, 2020, now Pat. No. 11,523,785.

(51) Int. Cl. A61B 6/00; A61B 6/03; A61B 6/46

(52) U.S. Cl. CPC A61B 6/4441; A61B 6/032; A61B 6/466; A61B 6/54

(57) ABSTRACT

A medical imaging system includes a movable station having a C-arm, a collector, an X-ray beam emitter, and a controller. The collector is attached to a first end of the C-arm. The X-ray beam emitter faces the collector to emit an X-ray beam in a direction of the collector and is attached to a second end of the C-arm. The controller moves one of the X-ray beam emitter and the collector to a first offset position along a lateral axis orthogonal to the arc, and obtains a first set of images by rotating the collector and the X-ray beam emitter along the arc about a scanned volume. The controller moves the one of the X-ray beam emitter and the collector to a second offset position along the lateral axis, and obtains a second set of images. The controller combines the first and second set of images to generate a three-dimensional image of the scanned volume.

### CROSS-REFERENCE TO RELATED APPLICATIONS

This application is a continuation of U.S. patent application Ser. No. 18/393,847, filed on Dec. 22, 2023, which is a continuation of U.S. patent application Ser. No. 18/055,509, filed Nov. 15, 2022 (published as U.S. Pat. Pub. No. 2023-0079430), which is a continuation of U.S. patent application Ser. No. 17/030,581 filed Sep. 24, 2020, now U.S. Pat. No. 11,523,785, which is incorporated by reference herein in its entirety for all purposes.

### TECHNICAL FIELD

The present disclosure relates to medical imaging systems, and more particularly, controlled movement of the imaging system or components thereof.

### BACKGROUND OF THE DISCLOSURE

Healthcare practices have shown the tremendous value of three-dimensional imaging such as computed tomography (CT) imaging, as a diagnostic tool in the Radiology Department. These imaging systems generally contain a fixed bore into which the patient enters from the head or foot. Other areas of care, including the operating room, intensive care departments and emergency departments, rely on two-dimensional imaging (fluoroscopy, ultrasound, 2-D mobile X-ray) as the primary means of diagnosis and therapeutic guidance.

While mobile solutions for ‘non-radiology department’ and patient-centric 3-D imaging do exist, they are often limited by their freedom of movement to effectively position the system without moving the patient. Their limited freedom of movement has hindered the acceptance and use of mobile three-dimensional imaging systems.

Therefore, there is a need for a small scale and/or mobile three-dimensional imaging systems for use in the operating room, procedure rooms, intensive care units, emergency departments and other parts of the hospital, in ambulatory surgery centers, physician offices, and the military battlefield, which can access the patients in any direction or height and produce high-quality three-dimensional images. These imaging systems may include intra-operative CT and magnetic resonance imaging (MRI) scanners, and robotic systems to aid in their use or movement. These include systems with 180-degree movement capability (“C-arms”) and may also include imaging systems with 360-degree movement capability (“O-arms”).

Some three-dimensional imaging systems expose patients to substantial non-uniform radiation dosages, which affects imaging clarity, and may require longitudinal movements of the imaging device to separately image different sections of the desired volumes and then use computationally complex operations to attempt to stitch together those scan volumes. A related complication when attempting to image spaced scan volumes is how to precisely reposition the imaging system. Image stitching can be especially difficult in an operating room or operating theatre, where the size and weight of the imaging equipment and the presence of numerous required personnel make it difficult to precisely reposition the imaging equipment.

### SUMMARY OF THE DISCLOSURE

Some embodiments of the present disclosure are directed to a medical imaging system that includes a movable station having a C-arm, an X-ray collector, an X-ray beam emitter, and a controller. The C-arm has a first end and a second end that are movable along an arc relative to the movable station. The collector is attached to the first end of the C-arm. The X-ray beam emitter faces the collector to emit an X-ray beam in a direction of the collector and is attached to the second end of the C-arm. The controller is configured to move one of the X-ray beam emitter and the collector to a first offset position relative to each other along a lateral axis orthogonal to the arc. The controller obtains a first set of images by rotating the collector and the X-ray beam emitter along the arc about a scanned volume while the X-ray beam emitter and the collector are positioned with the first offset position. The controller moves the one of the X-ray beam emitter and the collector to a second offset position relative to each other along the lateral axis orthogonal to the arc. The controller obtains a second set of images by rotating the collector and the X-ray beam emitter along the arc about the scanned volume while the X-ray beam emitter and the collector are positioned with the second offset position. The controller combines the first and second set of images to generate a three-dimensional image of the scanned volume.

Some other embodiments of the present disclosure are directed to another medical imaging system that includes a movable station having a C-arm, a collector, two X-ray beam emitters, and a controller. The C-arm has a first end and a second end that are movable along an arc relative to the movable station. The collector is attached to the first end of the C-arm. A first X-ray beam emitter and a second X-ray beam emitter both face the collector to emit X-ray beams in a direction of the collector and both are attached to the second end of the C-arm. The collector and the first and second X-ray beam emitters are spaced apart relative to each other along a lateral axis orthogonal to the arc. The controller is configured to obtain at least one set of images by rotating the collector and the first and second X-ray beam emitters along the arc about a scanned volume, and to combine the at least one set of images to generate a three-dimensional image of the scanned volume.

Some other embodiments of the present disclosure are directed to a computer program product including a non-transitory computer readable medium storing program code executable by at least one processor of a controller of a medical imaging system to perform operations for X-ray imaging. The operations move one of an X-ray beam emitter and a collector to a first offset position relative to each other along a lateral axis orthogonal to an arc. The operations obtain a first set of images by rotating the collector and a X-ray beam emitter along the arc about a scanned volume while the X-ray beam emitter and the collector are positioned with the first offset position. The operations move the one of the X-ray beam emitter and the collector to a second offset position relative to each other along the lateral axis orthogonal to the arc. The operations obtain a second set of images by rotating the collector and the X-ray beam emitter along the arc about the scanned volume while the X-ray beam emitter and the collector are positioned with the second offset position. The operations combine the first and second set of images to generate a three-dimensional image of the scanned volume.

It is noted that aspects described with respect to one embodiment disclosed herein may be incorporated in different embodiments although not specifically described relative thereto. That is, all embodiments and/or features of any embodiments can be combined in any way and/or combination. Moreover, methods, systems, and/or computer program products according to embodiments will be or become apparent to one with skill in the art upon review of the following drawings and detailed description. It is intended that all such additional methods, systems, and/or computer program products be included within this description and protected by the accompanying claims.

### BRIEF DESCRIPTION OF DRAWINGS

Aspects of the present disclosure are illustrated by way of example and are not limited by the accompanying drawings. In the drawings:

FIG. 1 illustrates components of a traditional imaging system including an X-ray beam emitter emitting a beam to a collector rotating about a patient on a rotation axis.

FIG. 2 illustrates a 3D volume created by performing an image scan while rotating about a fixed axis and back projecting the obtained set of images.

FIG. 3 illustrates substantial non-uniform radiation dosages of the imaging volume.

FIG. 4 illustrates a Cone Beam Computed Tomography, CBCT, scan volume.

FIG. 5 illustrates a configuration of collimators to limit the X-rays to a triangular strip of interest for acquiring a scan volume such as shown in the earlier figures.

FIG. 6 illustrates the X-ray beam emitter lateral offset in a first direction relative to the collector of a medical imaging system while performing an image scan to generate a first set of images according to some embodiments of the present disclosure.

FIG. 7 illustrates a reconstruction of the image volume that may be created by the medical imaging system configured according to FIG. 6.

FIG. 8 illustrates a two-dimensional side perspective of the image volume created by the medical imaging system configured according to FIG. 6.

FIG. 9 illustrates the X-ray beam emitter lateral offset in an opposite second direction relative to the collector of the medical imaging system while performing an image scan to generate a second set of images according to some embodiments of the present disclosure.

FIG. 10 illustrates a reconstruction of the image volume that may be created by the medical imaging system configured according to FIG. 9.

FIG. 11 illustrates a two-dimensional side perspective of the image volume created by the medical imaging system configured according to FIG. 9.

FIG. 12 illustrates a two-dimensional side perspective of the image volume created by the combination of the first and second set of scan volumes of FIGS. 9 and 12 in accordance with some embodiments of the present disclosure.

FIG. 13 illustrates traditionally configured longitudinally offset image volumes that are stitched together, end to end.

FIG. 14 illustrates traditionally configured image volumes stitched together with the corners of the 2x regions opposed to one another.

FIG. 15 illustrates an example configuration of the X-ray beam emitter angled up while performing an image scan to generate a first set of images in accordance with some embodiments of the present disclosure.

FIG. 16 illustrates a reconstruction of the image volume created by performing an image scan with the X-ray beam emitter angled up as shown in FIG. 15.

FIG. 17 illustrates a two-dimensional side perspective of an image volume created by the medical imaging system configured as shown in FIG. 15.

FIG. 18 illustrates an example configuration of the X-ray beam emitter angled down while performing a second image scan to generate a second set of images in accordance with some embodiments.

FIG. 19 illustrates a reconstruction of the image volume created by the medical imaging system when configured as shown in FIG. 18.

FIG. 20 illustrates a two-dimensional side perspective of the volume created by the medical imaging system when configured as shown in FIG. 18.

FIG. 21 illustrates the two-dimensional side perspective of the volume created by the combination of the first and second set of images from the image scans of FIGS. 16-18 and then FIGS. 18-20.

FIG. 22 illustrates the X-ray beam emitter configured to be moveable along a linear rail attached to the second end of the C-arm in accordance with some embodiments of the present disclosure.

FIG. 23 illustrates the X-ray beam emitter configured to be moveable about an angular pivot attached to the second end of the C-arm in accordance with some embodiments of the present disclosure.

FIG. 24 illustrates the medical imaging system utilizing two fixed emitters with a single collector, in accordance with some embodiments of the present disclosure.

FIG. 25 illustrates a medical imaging system utilizing three fixed X-ray beam emitters with a single collector, in accordance with some embodiments of the present disclosure.

FIG. 26 illustrates three views of the collector and X-ray beam emitter rotated +9.4 degrees, 0 degrees, and −9.4 degrees.

FIGS. 27a-d illustrate four views of the C-arm rotating the collector and X-ray beam emitter along an arc while the collector and X-ray beam emitter are rotated +9.4 degrees distal to the other rotations during the spin.

FIGS. 28a-d illustrate four view of the C-arm rotating the collector and X-ray beam emitter along an arc while the collector and X-ray beam emitter are rotated −9.4 degrees distal to the other rotations during the spin.

FIG. 29 illustrates the collector in a first laterally offset position in accordance with some embodiments of the present disclosure.

FIG. 30 illustrates the collector in a second laterally offset position in accordance with some embodiments of the present disclosure.

FIG. 31 illustrates a block diagram of components of a medical imaging system configured in accordance with some embodiments of the present disclosure.

FIG. 32 and FIG. 33 are flowcharts of operations by a controller of a medical imaging system in accordance with some embodiments of the present disclosure.

### DETAILED DESCRIPTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

For purposes of this application, the terms “code”, “software”, “program”, “application”, “software code”, “software module”, “module” and “software program” are used interchangeably to mean software instructions that are executable by a processor. A “user” can be a physician, nurse, or other medical professional.

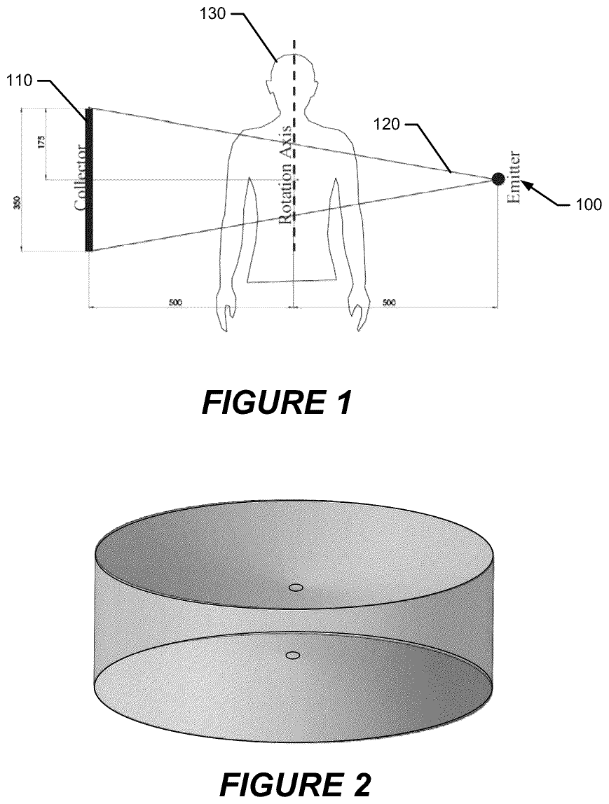

Turning now to the drawing, FIG. 1 illustrates components of a traditional imaging system including an X-ray beam emitter 100 emitting a beam 120 to a collector 110 rotating about a patient 130 on a rotation axis. The X-ray beam emitter 100 is mounted to one end of a C-arm while an imaging sensor such as the collector 110 is mounted to the other end of the C-arm and faces the X-ray beam emitter. A motor attached to a vertical shaft of the C-arm is designed to rotate the collector 110 and X-ray beam emitter 100 up to 360 degrees about the rotational axis under the control of a controller. In this example, the X-ray beam emitter 100 transmits an X-ray beam 120 which is received by collector 110 after passing through a relevant portion of a patient 130.

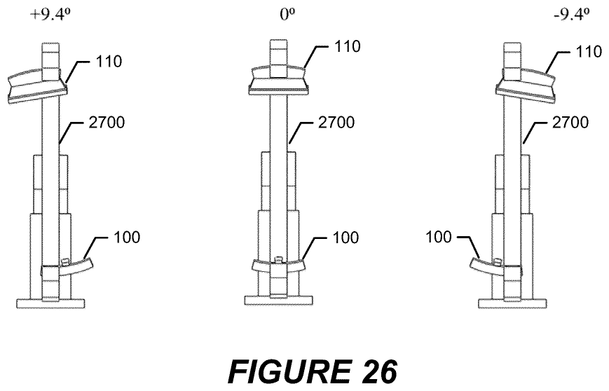

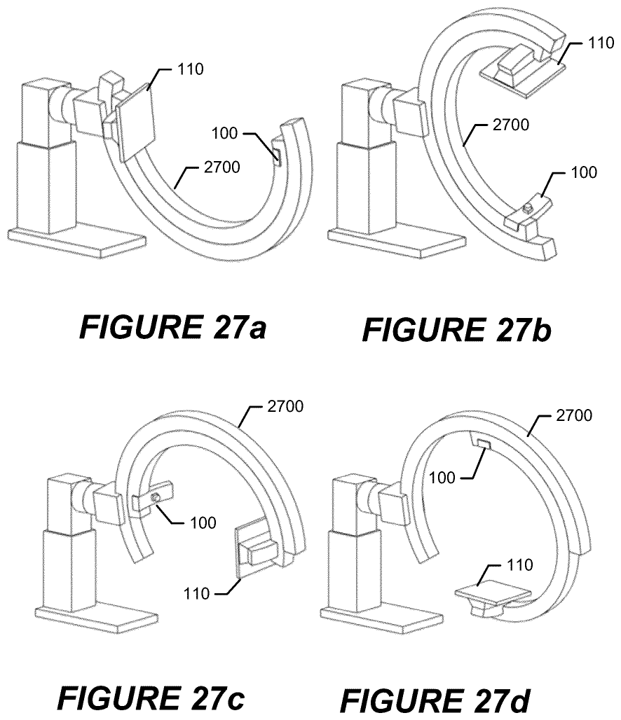

An example C-arm including an X-ray emitter and collector is illustrated FIGS. 26, 27a-d, and 28-a-d.

It may be desirable to take X-rays of patient 130 from a number of different positions, without the need for frequent manual repositioning of patient 130 which may be required in an X-ray system. The medical imaging system may be in the form of a C-arm that includes an elongated C-shaped member terminating in opposing distal ends of the “C” shape. The space within C-arm of the arm may provide room for the physician to attend to the patient substantially free of interference from X-ray support structure.

The image capturing components include the X-ray beam emitter 100 and the collector 110, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of patient 130 to be acquired from multiple directions or in multiple planes.

The images collected while the C-arm rotates the collector 110 and X-ray beam emitter 100 around the patient 130 are then combined to generate a three-dimensional image of a scanned volume. When a 3D reconstruction from a cone beam 120 is produced, the volume is limited by what can be captured by the X-ray path. For example, a collector may be 35 cm across and spacing between emitter and collector may be 1 m, such as shown in FIG. 1.

FIG. 2 illustrates a 3D image volume created by performing an image scan while rotating about a fixed axis and back projecting the obtained set of images. Because of how the cone beam, which is triangular in cross-section, is swept about the rotation axis 130, this 3D image volume constructed by back projecting is generally cylindrical in shape, but with inward facing conical voids at the top and bottom ends of the cylinder, which is where x-ray beams do not pass.

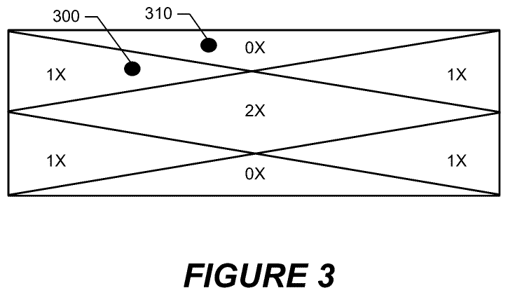

FIG. 3 illustrates a two-dimensional side perspective of the image volume illustrated in FIG. 2. Some parts of the patient anatomy toward the edges are only penetrated by X-rays during certain perspectives. Therefore, the image reconstruction is less robust. For example, anatomy located at 300 is not struck by X-rays from left to right but is struck from right to left. Other anatomy is never intersected, such as anatomy located at point 310. The regions of FIG. 3 are labeled 0x, 1x, or 2x, meaning they are intersected by X-rays not at all (0x), not from all perspectives (1x) or from all perspectives (2x). Unnecessary and/or inefficient use of radiation should be avoided when imaging a patient, so regions not intersected by X-rays or not intersected from all perspectives should try to be avoided or minimized.

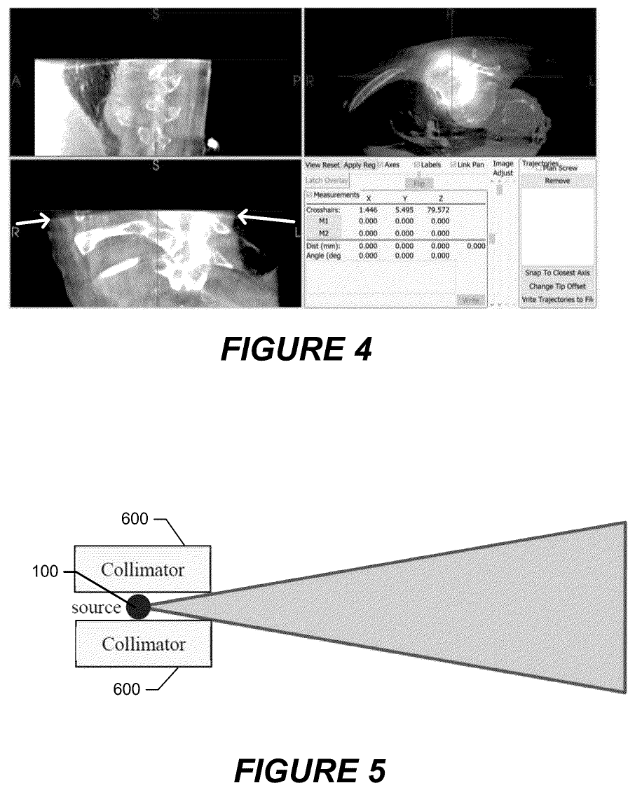

FIG. 4 illustrates an example cone beam computed tomography, CBCT, scan volume. Note the white halo on the axial view where the scan reaches its limit of visibility of the scanned anatomy, and simultaneously, the other views show the darker regions where tissues are intersected from only a subset of perspectives. White lines in the bottom left image are overlaid where the boundary from 1x to 2x as described in FIG. 3 occurs.

Because the region of interest is only in the “2X” area, the height of the cylindrical scan volume is reduced compared to the height of the collector. In the illustrated example, the height of the 2X region is 175 mm, which is 50% of the collector height.

CBCT devices place a metal (lead) barrier or collimator around the emitter to prevent excessive irradiation of the patient. However, the collimation occurs on the side of the emitter, so unwanted X-rays still extend above and below the region of interest. For example, FIG. 5 illustrates a configuration of collimators 600 to limit the X-rays to triangular strip of interest for acquiring the scan volume of the earlier figures.

The X-ray beam emitter contains a collimator 600 that forms a window which shapes an X-ray beam transmitted therethrough toward the collector. The collimator 600 is configured to move a location of the widow in a lateral direction across the arc direction. A controller is configured to control movement of the window by the collimator 600 to steer the X-ray beam laterally across the collector in accordance with some embodiments.

In some embodiments, a collimator 600 is used to adjust the size and/or lateral location of the X-ray beam, e.g., relative to the direction of the arcuate image scan.

The collimator 600 includes a pair of shutters (not shown) that are on opposite sides of a window, in accordance with some embodiments. The shutters are formed from a material, such as lead, that substantially blocks X-rays. Motor mechanisms are connected to slide a respective one of the shutters along tracks extending in the lateral direction to change the locations of opposing edges of the window. The controller controls the motor mechanisms to set the lateral distance between the shutters to control width of the X-ray beam, and further controls the motor mechanisms to move the window to steer the X-ray beam across the collector.

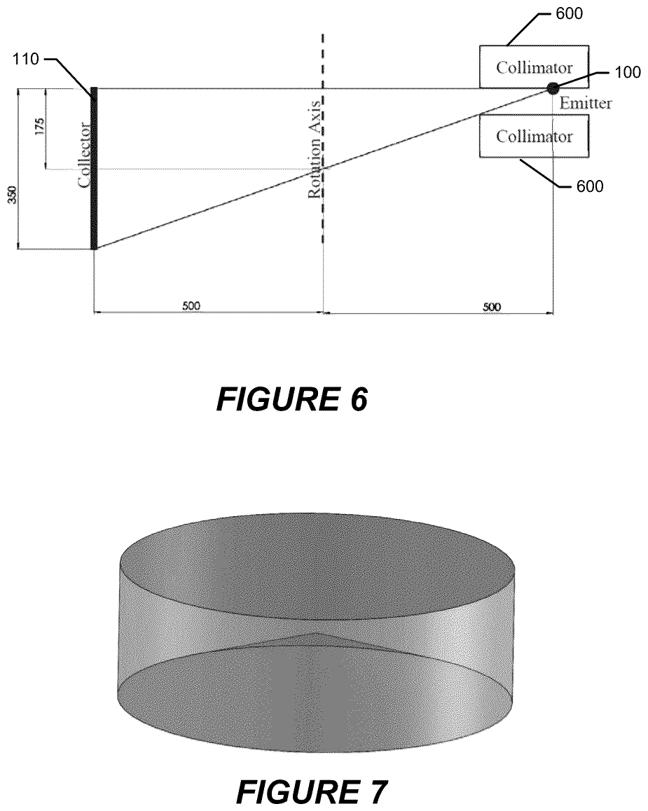

FIG. 6 illustrates the X-ray beam emitter 100 positioned laterally offset in a first direction relative to the collector 110 of a medical imaging system to be roughly in line across the x-ray path with the top of the collector while being rotated about the rotational axis along an arc to perform an image scan to generate a first set of images according to some embodiments of the present disclosure.

FIG. 7 illustrates the reconstruction of the image volume that may be created by the image scan performed while the medical imaging system is configured as shown in FIG. 6. Because of how the cone beam, which is triangular in cross-section, is swept about the rotation axis 130, this 3D image volume constructed by back projecting is generally cylindrical in shape, but with one inward facing conical void at the bottom end of the cylinder, which is where x-ray beams do not pass.

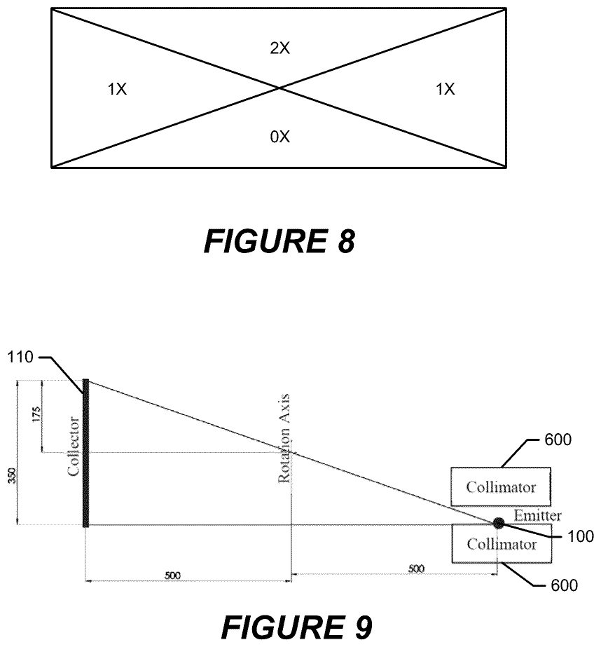

FIG. 8 illustrates the two-dimensional side perspective of the image volume created by the image scan performed while the medical imaging system is configured as shown in FIG. 6. As with the case where the emitter is centered (FIG. 3) instead of laterally offset, there are regions of 0x, 1x, and 2x; however, with the emitter offset in a first direction, these regions are differently spatially distributed than in the centered case.

FIG. 9 illustrates the X-ray beam emitter 100 laterally offset in an opposite second direction relative to the collector 110 of the medical imaging system to be roughly in line across the x-ray path with the bottom of the collector while performing an image scan to generate a second set of images according to some embodiments of the present disclosure.

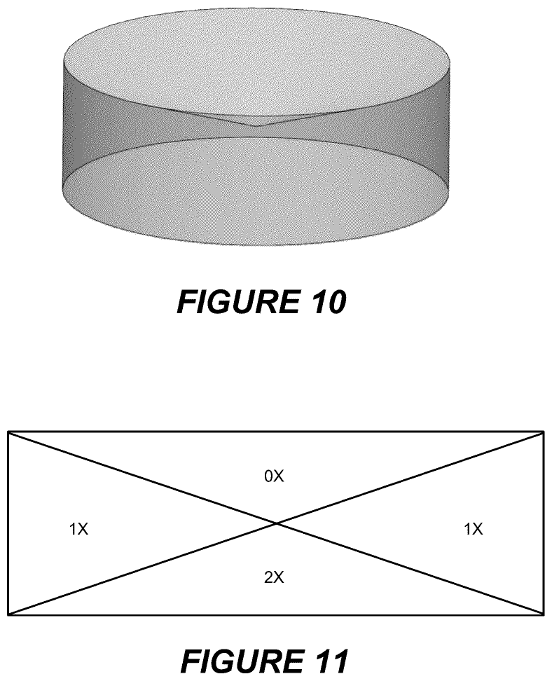

FIG. 10 illustrates the reconstruction of the image volume that may be created by the image scan performed while the medical imaging system is configured according to FIG. 9. Because of how the cone beam, which is triangular in cross-section, is swept about the rotation axis 130, this 3D image volume constructed by back projecting is generally cylindrical in shape, but with one inward facing conical void at the top end of the cylinder, which is where x-ray beams do not pass.

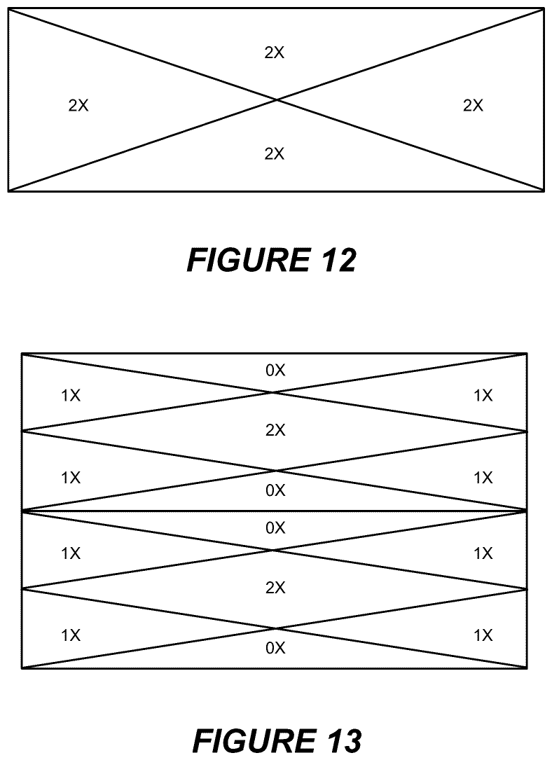

FIG. 11 illustrates the two-dimensional side perspective of the image volume created by the image scan performed while the medical imaging system is configured according to FIG. 9. As with the case where the emitter is positioned centered (FIG. 3) instead of laterally offset, there are regions of 0x, 1x, and 2x; however, with the emitter offset in a second direction, these regions are differently spatially distributed than in the centered case and inversely to the case where the emitter is offset in a first direction (FIG. 8).

FIG. 12 illustrates a two-dimensional side perspective of the image volume created by the combination (overlap) of the first and second set of scan volumes of FIGS. 9 and 12 in accordance with some embodiments of the present disclosure. The image volume is cylindrical with the same height as the height of the collector 110. Note how consistent the dosage is in every part of the scan (2x). The regions in all areas are penetrated by X-rays from all perspectives. There are no 1x regions at the top or bottom of the cylinder, in sharp contrast to the image volumes shown in FIGS. 7 and 10. This uniformity is because the top and bottom regions are penetrated at 2x by the individual scans and the regions to left and right are penetrated at 1x each, so when summed, they all penetrate at 2x.

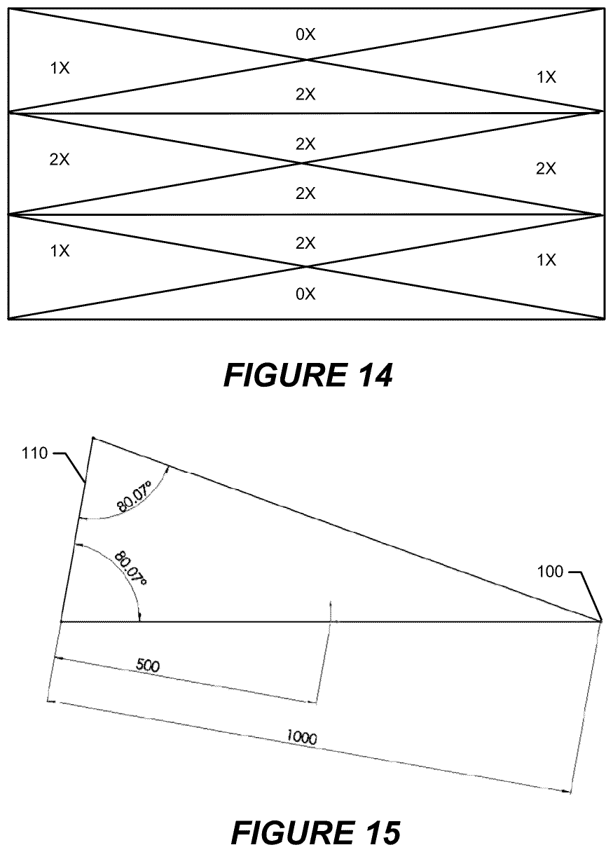

For comparison with the above embodiment, consider taking two longitudinally offset image volumes according to the traditional configuration of the X-ray beam emitter 100 centered on the collector 110 (as illustrated in FIG. 1) and stitching them together, end to end. FIG. 13 illustrates these traditionally configured longitudinally offset image volumes stitched together, end to end. The image volumes cannot be stitched exactly end to end because of the suboptimal X-ray penetration conditions at the ends of each volume. Instead, the corners of the 2x regions would need to be opposed to one another, as illustrated in FIG. 14. In this case, the combined image volume has the same working height as in the modified method, but there is less consistent penetration of all regions and there is unwanted and unused penetration of X-rays beyond either end of the image volume, which means the patient would receive a greater X-ray dose if images acquired by the traditional configuration were stitched than if some embodiments of the present disclosure were used. Although stacking images end to end would result in an image twice as long, it would require the entire X-ray apparatus (collector and X-ray beam emitter) to be moved longitudinally by ½ the collector height, in contrast with some embodiments of the present disclosure, in which only the emitter is moved.

In some other embodiments of the present disclosure, the X-ray beam emitter and collector can be angled up and down during image scans. FIG. 15 illustrates an example configuration of the X-ray beam emitter 100 angled up while performing an image scan to generate a first set of images in accordance with some embodiments of the present disclosure.

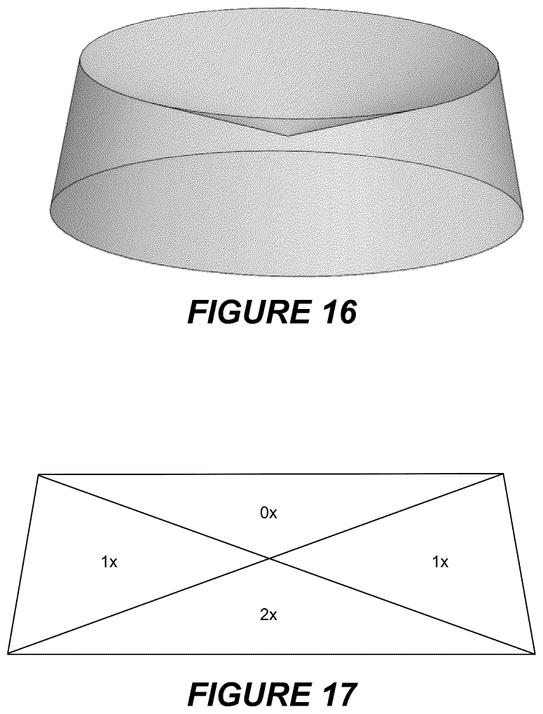

FIG. 16 illustrates the reconstruction of the image volume created by performing an image scan with the X-ray beam emitter angled up as shown in FIG. 15. This image volume is shaped like a pet food bowl—flat on the bottom and tapered on the sides, with a conical void on the top where x-rays do not pass.

FIG. 17 illustrates the two-dimensional side perspective of the image volume created by the medical imaging system configured as shown in FIG. 15.

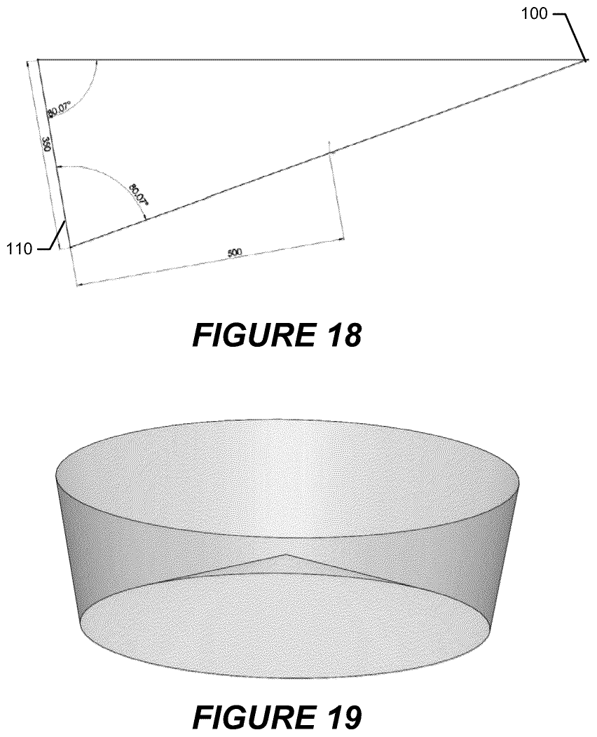

FIG. 18 illustrates an example configuration of the X-ray beam emitter angled down while performing a second image scan to generate a second set of images in accordance with some embodiments.

FIG. 19 illustrates the reconstruction of the image volume created by the medical imaging system when configured as shown in FIG. 18. The 3D shape of this image volume is the inverse of that shown in FIG. 16.

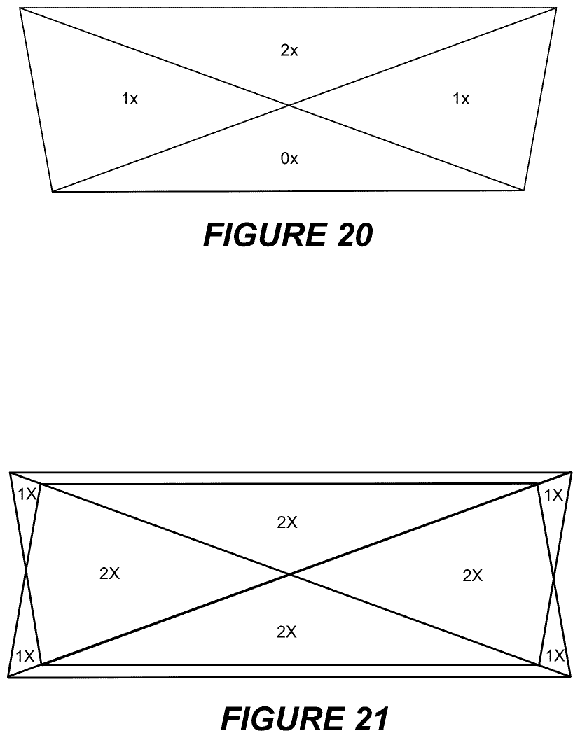

FIG. 20 illustrates the two-dimensional side perspective of the volume created by the medical imaging system when configured as shown in FIG. 18.

FIG. 21 illustrates the two-dimensional side perspective of the volume created by the combination of the first and second set of images from the image scans of FIGS. 16-18 and then FIGS. 19-21. The combined volumes slightly exceed the collector height. In this example, the angle difference is 19.86°, so just tilting the emitter-collector assembly up 9.4° and down 9.4° doubles the image volume. Combination of volumes in this embodiment can provide similar advantages over the stitching of 2 standard images: there is less unused radiation at either end of the scan and the volume is essentially doubled with no longitudinal movement of the machine, just a slight angulation.

In various embodiments of the present disclosure, a different method can be used to generate CBCT reconstructions by: (1) moving the emitter to two or more different offset locations while keeping the collector in a fixed location; (2) using 2 emitters separated by the height of the collector and phasing them; (3) moving the emitter and collector to two or more different perspective angles; or (4) moving the collector in a first direction and C-arm unit in a second (opposite) direction to two or more different offset positions while keeping the emitter fixed relative to the unit and the anatomical scan region fixed relative to the patient.

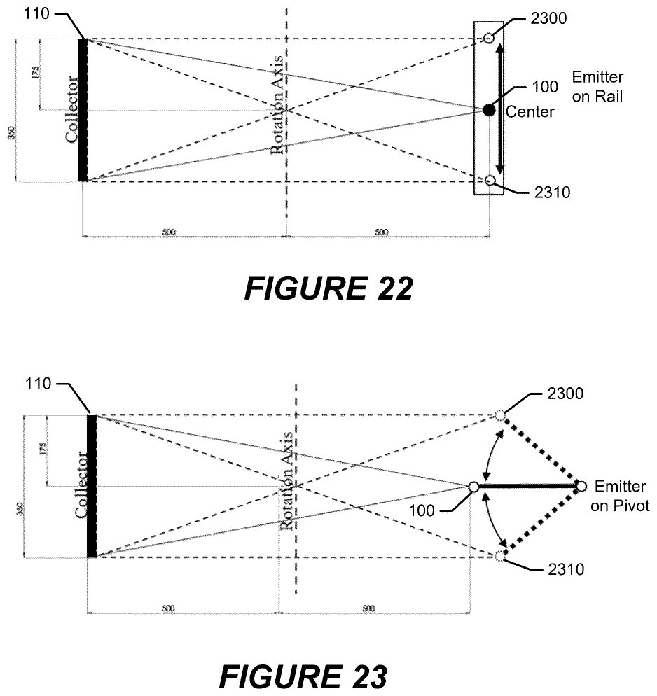

In some embodiments of the present disclosure, the X-ray beam emitter can be moved in an offset direction to one or more additional position other than the two positions illustrated in FIGS. 6, 9, 15, and 18 and additional scan volumes collected and combined with the scans already combined. For example, three scans can be collected in each of three emitter positions shown in FIG. 22. By further combining additional positions, the image quality may be improved and transitional regions represented by the diagonal lines on FIGS. 12 and 21 may have less artifact. Additional scans may be obtained at lower X-ray power to avoid unnecessarily dosing the patient.

In some embodiments, the medical imaging system is contemplated that has an X-ray beam emitter 100 on a moving platform to shift it to be in line with either the top, middle, or bottom of the collector 110. FIG. 22 illustrates the mechanism moving the X-ray beam emitter on a linear rail. FIG. 23 illustrates the mechanism moving (angularly rotating) the X-ray beam emitter on a pivot. If positioned at the center position, the system would operate traditionally. In the first offset position 2300 and the second offset position 2310, two separate scans would be taken and would be combined for a double-length scan volume.

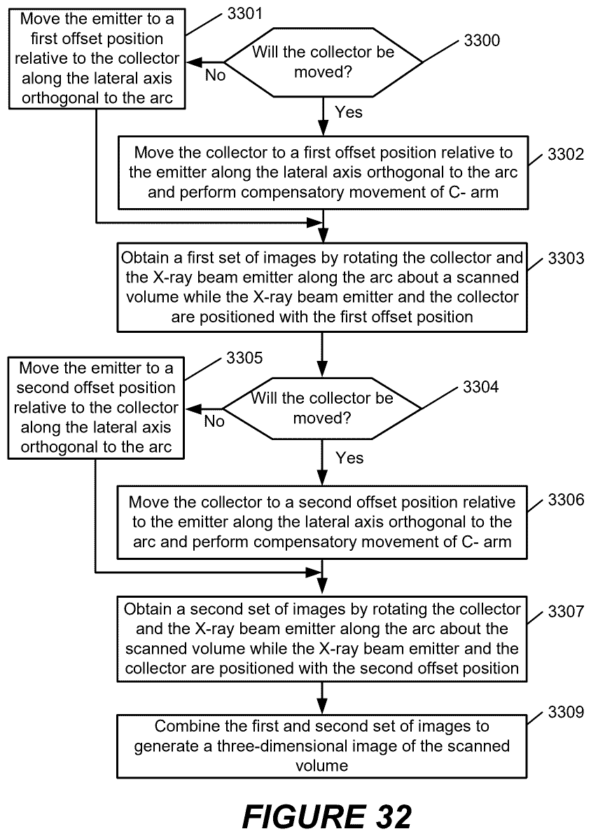

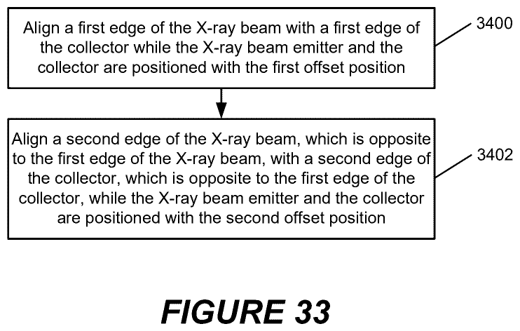

FIG. 32 and FIG. 33 illustrate flowcharts of operations by a controller in accordance with some embodiments of the present disclosure.

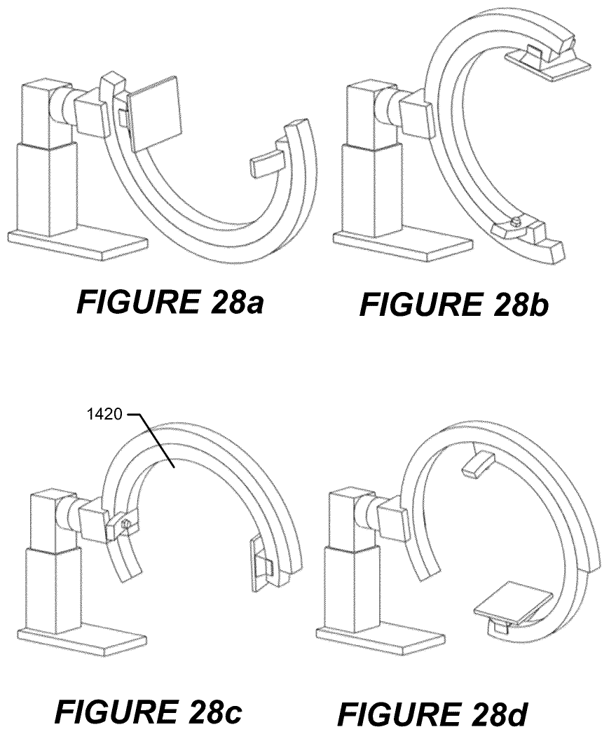

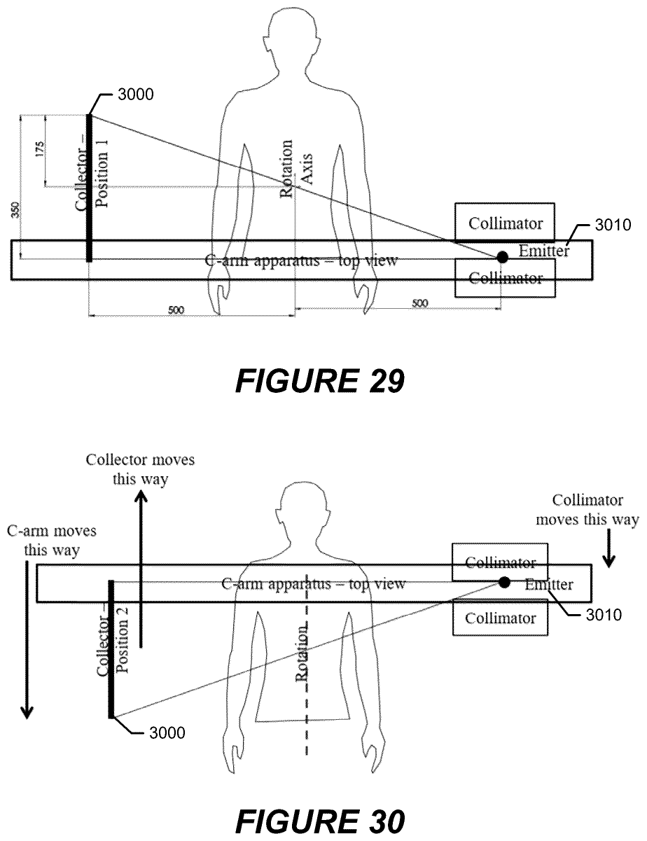

With reference to FIGS. 23, 23, and 32, some embodiments of the present disclosure are directed to a medical imaging system that includes a movable station having a C-arm, a collector 110, an X-ray beam emitter 100, and a controller. The C-arm has a first end and a second end that are movable along an arc relative to the movable station. The collector is attached to the first end of the C-arm. The X-ray beam emitter 100 faces the collector 110 to emit an X-ray beam 120 in a direction of the collector 110 and attached to the second end of the C-arm. A decision 3300 is made whether the collector 110 is to be moved to a first offset position 2300 for imaging. When the decision 3300 is made to not move the collector 110, the emitter 100 is moved 3301 to a first offset position 2300 relative to the collector 110 along the lateral axis orthogonal to the arc. If the emitter 100 moves relative to the collector 110 and C-arm, no movement of the C-arm is required. In contrast, when the decision 3300 is made to move the collector 110, the collector 110 is moved to a first offset position 2300 relative to the emitter 100 along the lateral axis orthogonal to the arc. If the collector 110 moves relative to the emitter 100 and C-arm, a compensatory movement of the C-arm is required to keep the anatomical region of interest centered relative to the collector 110. This compensatory movement is illustrated in FIGS. 28 and 29. The controller obtains 3303 a first set of images by rotating the collector and the X-ray beam emitter 100 along the arc about a scanned volume while at least one of the X-ray beam emitter 100 and the collector 110 is positioned with the first offset position 2300.

A second decision 3304 is made whether the collector 110 is to be moved to a second offset position 2310 for imaging. When the decision 3304 is made to not move the collector 110, the emitter 100 is moved 3305 to a second offset position 2310 relative to the collector 110 along the lateral axis orthogonal to the arc. In contrast, when the decision 3300 is made to move the collector 110, the collector 110 is moved to a second offset position 2310 relative to the emitter 100 along the lateral axis orthogonal to the arc. If the collector 110 moves relative to the emitter 100 and C-arm, a compensatory movement of the C-arm is required to keep the anatomical region of interest centered relative to the collector 110. This compensatory movement is illustrated in FIGS. 29 and 30. The controller obtains 3306 a second set of images by rotating the collector 110 and the X-ray beam emitter 100 along the arc about the scanned volume while at least one of the X-ray beam emitter 100 and the collector 110 is positioned with the second offset position 2310. The controller combines 3308 the first and second sets of images to generate a three-dimensional image of the scanned volume.

With further reference to FIG. 33, in some embodiments the controller is further configured to align 3400 a first edge of the X-ray beam with a first edge of the collector 110 while the X-ray beam emitter 100 and the collector 110 are positioned with the first offset position 2300. The controller is also configured to align 3402 a second edge of the X-ray beam, which is opposite to the first edge of the X-ray beam, with a second edge of the collector 110, which is opposite to the first edge of the collector 110, while the X-ray beam emitter 100 and the collector are positioned with the second offset position 2310.

In some embodiments, the X-ray beam emitter 100 is moveable along a linear rail attached to the second end of the C-arm, as illustrated in FIG. 22. The controller is further configured to move the X-ray beam emitter 100 along the linear rail between the first offset position 2300 and second offset position 2310 relative to the collector 110. The X-ray beam emitter 100 can include a collimator that forms a window through which the X-ray beam is emitted toward the collector 110, where the collimator is configured to move a widow along the lateral axis. The controller is further configured to control movement of the window by the collimator to compensate for movement of the X-ray beam emitter 100 along the linear rail between the first offset position 2300 and second offset position 2310 to provide alignment of the X-ray beam with the collector 110.

The controller can be further configured to determine the first offset position 2300 and second offset position 2310 for the X-ray beam emitter 100 based on levels of scatter and transmission of the X-ray beam detected in images obtained from the collector 100 while the X-ray beam emitter is moved to each of the first offset position 2300 and second offset position 2310.

In some embodiments, the X-ray beam emitter 100 is moveable (angularly rotated) about an angular pivot attached to the second end of the C-arm, as illustrated in FIG. 23. The controller is further configured to angularly rotate the X-ray beam emitter 100 about the angular pivot between the first offset position 2300 and second offset position 2310 relative to the collector 110.

The controller can be further configured to determine the first offset position 2300 and second offset position 2310 for the X-ray beam emitter 100 based on levels of scatter of the X-ray beam detected in images obtained from the collector 110 while the X-ray beam emitter 100 is angularly rotated about the angular pivot between the first offset position 2300 and second offset position 2310.

In some embodiments, a collimator forms a window through which the X-ray beam is emitted toward the collector 110, and the collimator is configured to move a widow along the lateral axis. The controller is further configured to control movement of the window by the collimator to compensate for rotation of the X-ray beam emitter 100 about the angular pivot between the first offset position 2300 and second offset position 2310 to provide alignment of the X-ray beam with the collector 110.

Note that on the pivoting emitter setup, the emitter is closer to the collector at the traditional, centered position than at the first offset position 2300 and second offset position 2310. The magnitude of this difference depends on the length of the pivot arm. As long as the distance from the emitter to collector at the first offset position 2300 and second offset position 2310 are the same, the shapes of the volumes obtained from scans should be exactly inverse and when combined should form uniform double-length scans.

In some example embodiments, the C-arm spins the collector 110 and the X-ray beam emitter 100 at 30 degrees per second. One projection is used to produce an image per degree. An X-ray pulse of 10 ms is used to produce an image. Then back projection computing is used to weight the areas that overlap to combine the two sets of images.

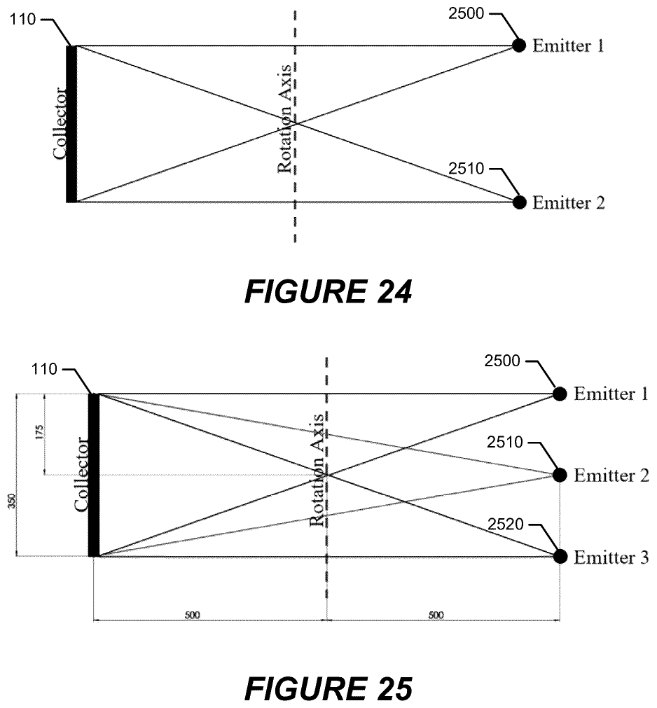

In some embodiments, the medical imaging system utilizes two fixed emitters with a single collector, as illustrated in FIG. 24. The medical imaging system includes a movable station having a C-arm, a collector 110, two X-ray beam emitters 2500 and 2510, and a controller. The C-arm has a first end and a second end that are movable along an arc relative to the movable station. The collector 110 is attached to the first end of the C-arm. The first X-ray beam emitter 2500 and a second X-ray beam emitter 2510 both face the collector 110 to emit X-ray beams in a direction of the collector 110 and both attached to the second end of the C-arm. The collector 110 and the first and second X-ray beam emitters 2500 and 2510 are spaced apart relative to each other along a lateral axis orthogonal to the arc. The controller is configured to obtain at least one set of images by rotating the collector and the first and second X-ray beam emitters along the arc about a scanned volume, and to combine the at least one set of images to generate a three-dimensional image of the scanned volume.

In some of these embodiments, the X-ray beams emitted by the first and second X-ray beam emitters 2500 and 2510 are aligned with the collector 110 while the controller obtains the at least one set of images.

In some embodiments the image volume generated from X-ray images using both X-ray beam emitters 2500 and 2510 are captured separately. The C-arm is spun once with the first X-ray beam emitter 2500 activated while capturing a first set of images, and the C-arm is then spun again with the second X-ray beam emitter 2510 activated while capturing a second set of images. The two sets of images are then combined.

In these embodiments, the controller is further configured to activate the first X-ray beam emitter 2500 to emit X-rays while maintaining the second X-ray beam emitter 2510 deactivated and while obtaining a first set of images by rotating the collector 110 and the first and second X-ray beam emitters 2500 and 2510 along the arc about a scanned volume. The controller is also configured to activate the second X-ray beam emitter 2510 to emit X-rays while maintaining the first X-ray beam emitter 2500 deactivated and while obtaining a second set of images by rotating the collector 110 and the first and second X-ray beam emitters 2500 and 2510 along the arc about the scanned volume. The controller is also configured to combine the first and second set of images to generate the three-dimensional image of the scanned volume.

In some embodiments, the image volume generated from X-ray images using both X-ray beam emitters 2500 and 2510 are captured by alternating phasing between X-ray beam emitters 2500 and 2510. The controller rapidly switches between the first X-ray beam emitter 2500 and the second X-ray beam emitter 2510 during one spin of the C-arm while generating two sets of images (one set from the first X-ray beam emitter 2500 and the other set from the second X-ray beam emitter 2510). The two sets of images are then combined.

In these dual emitter embodiments, the controller can be further configured to repetitively alternate between activation of the first X-ray beam emitter 2500 to emit X-rays while maintaining the second X-ray beam emitter 2510 deactivated and while obtaining an image for a first set of images and activation of the second X-ray beam emitter 2510 to emit X-rays while maintaining the first X-ray beam emitter 2500 deactivated and while obtaining an image for a second set of images, while rotating the collector 110 and the first and second X-ray beam emitters 2500 and 2510 along the arc about the scanned volume. The controller is also configured to combine the first and second set of images to generate the three-dimensional image of the scanned volume.

In some embodiments, the controller is further configured to repetitively alternate between an activation of the first X-ray beam emitter 2500 the activation of the second X-ray beam emitter 2510 at no more than one degree increments along the arc while rotating the collector and the first and second X-ray beam emitters 2500 and 2510 about the scanned volume.

The controller can be further configured to repetitively alternate between the activation of the first X-ray beam emitter 2500 the activation of the second X-ray beam emitter 2510 at no more than half degree increments along the arc while rotating the collector and the first and second X-ray beam emitters 2500 and 2510 about the scanned volume.

In some other dual emitter embodiments, the image volume generated from X-ray images captured using both X-ray beam emitters 2500 and 2510 are captured by simultaneously activating both X-ray beam emitters 2500 and 2510. The X-ray data is collected on the same collector 110 from both X-ray beam emitters 2500 and 2510 in one spin. Then the back-projections from these combined images are computed.

In these embodiments, the controller is further configured to simultaneously activate the first and second X-ray beam emitters to emit X-rays while obtaining a set of images while rotating the collector and the first and second X-ray beam emitters along the arc about the scanned volume. The controller is further configured to combine the set of images to generate the three-dimensional image of the scanned volume.

FIG. 25 illustrates a medical imaging system utilizing three fixed X-ray beam emitters 2600, 2610, and 2620 with a single collector 110. In some of these embodiments, traditional centered imaging uses the second X-ray beam emitter 2610 while the first and third X-ray beam emitters 2600 and 2620 remain deactivated.

In some embodiments, the medical imaging system collects a “double long” image using the first and third X-ray beam emitters 2600 and 2620, phased, sequentially, or simultaneously activated with the second X-ray beam emitters 2610 always deactivated.

In some embodiments, the medical imaging system collects a “high resolution” image using the first, second, and third X-ray beam emitters 2600, 2610, and 2620, phased, sequentially, or simultaneously activated. This creates an image with the region in the center being penetrated twice as much as other scans.

In some embodiments, the medical imaging system has an additional axes and motors to produce an additional rotation distal to the other rotations, holding this offset angle during the spin. FIG. 26, FIG. 27, and FIG. 28 illustrate a medical imaging system having a C-arm 2700 with a controller configured to rotate the collector 110 and X-ray beam emitter 100 distal to the rotation of the C-arm 2700. FIG. 26 illustrates three views of the collector 110 and X-ray beam emitter 100 rotated +9.4 degrees, 0 degrees, and −9.4 degrees. FIGS. 27a-d illustrate four views of the C-arm rotating the collector 110 and X-ray beam emitter 100 along an arc while the collector 110 and the X-ray beam emitter are rotated +9.4 degrees distal to the other rotations during the spin. FIGS. 28a-d illustrate four views of the C-arm rotating the collector 110 and X-ray beam emitter 100 along an arc while the collector 110 and the X-ray beam emitter are rotated −9.4 degrees distal to the other rotations during the spin.

In some of these embodiments, the controller is further configured to angularly rotate the collector 110 and the X-ray beam emitter 100 toward each other in a first angular direction when the X-ray beam emitter 100 and the collector 110 are positioned with the first offset position 2300, and to angularly rotate the collector 110 and the X-ray beam emitter 100 toward each other in a second angular direction opposite to the first angular direction when the X-ray beam emitter 100 and the collector 110 are positioned with the second offset position 2310.

In some embodiments, FIGS. 29 and 30 illustrate a medical imaging system configured to include a movable collector 3000 and fixed X-ray beam emitter 3100. The collector 3000 has a linear bearing and motor, allowing it to move the collector 3000 to two different positions, in contrast to the embodiment of FIG. 22 which moved the X-ray beam emitter 3100 to two different positions and contrasted to the embodiment of FIG. 24 using multiple X-ray beam emitters. Moving the collector 3000 may involve moving the entire apparatus in a compensatory way (total movement=width of the collector plate) so that the collector 3000 via the apparatus movement remains in a fixed position relative to the patient. Additionally, collimators can be operated to move to different positions to allow the desired x-rays to reach the collector 3000 in its new position and block unused beams. FIG. 29 illustrates the collector 3000 in a first offset position. FIG. 30 illustrates the collector 3000 in a second offset position.

In some embodiments, the collector 3000 is moveable along a linear rail attached to the first end of the C-arm. The controller is further configured to move the collector 3000 along the linear rail between the first and second offset positions relative to the X-ray beam emitter 3010.

In some of the collector movement embodiments, the X-ray beam emitter 3010 comprises a collimator that forms a window through which the X-ray beam is emitted toward the collector 3000, wherein the collimator is configured to move a widow along the lateral axis. The controller is further configured to control movement of the window by the collimator to compensate for movement of the collector 3000 along the linear rail between the first and second offset positions to provide alignment of the X-ray beam with the collector 3000.

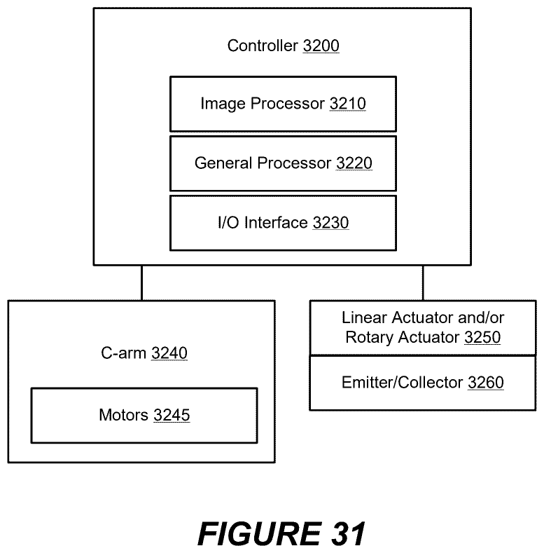

FIG. 31 illustrates a block diagram of components of a medical imaging system configured in accordance with some embodiments of the present disclosure. The medical imaging system includes a controller 3200, a C-arm 3240, a linear actuator and/or rotary actuator 3250 connected to an X-ray beam emitter or collector 3260. The controller 3200 includes an image processor 3210, a general processor 3220, and an I/O interface 3230. The image processor 3210 performs image processing to combine sets of images to generate a three-dimensional image of the scanned volume. The general processor 3220 is used to perform various embodiments of the present disclosure. The I/O interface 3230 communicatively couples the controller 3200 to other components of the medical imaging system. The C-arm 3240 includes motors 3245 used to move the collector and emitter along an arc, e.g., three hundred and sixty degrees, during image acquisition. Motors 3245 are controlled by C-arm the controller 3200. The controller 3200 can also control movement of the linear actuator and/or rotary actuator 3250.

### FURTHER DEFINITIONS AND EMBODIMENTS

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented in entirely hardware without software or may be a combination of hardware and software executed by a computer controller.

It is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

The corresponding structures, materials, acts, and equivalents of any means or step plus function elements in the claims below are intended to include any disclosed structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

What is claimed is:

1. A method for using a medical imaging system, said method comprising:
moving one of a X-ray beam emitter and a collector to a first offset position relative to each other along a lateral axis orthogonal to the arc;
obtaining a first set of images by moving the collector and the X-ray beam emitter about a scanned volume while the X-ray beam emitter and the collector are positioned with the first offset position;
moving the one of the X-ray beam emitter and the collector to a second offset position relative to each other along the lateral axis orthogonal to the arc;
obtaining a second set of images by moving the collector and the X-ray beam emitter about the scanned volume while the X-ray beam emitter and the collector are positioned with the second offset position; and
combining the first and second set of images to generate a three-dimensional image of the scanned volume.

2. The method of claim 1, further comprising:
aligning a first edge of the X-ray beam with a first edge of the collector while the X-ray beam emitter and the collector are positioned with the first offset position; and
aligning a second edge of the X-ray beam, which is opposite to the first edge of the X-ray beam, with a second edge of the collector, which is opposite to the first edge of the collector, while the X-ray beam emitter and the collector are positioned with the second offset position.

3. The method of claim 1, further comprising:
moving the X-ray beam emitter along the linear rail between the first and second offset positions relative to the collector.

4. The method of claim 3, wherein the X-ray beam emitter comprises a collimator that forms a window through which the X-ray beam is emitted toward the collector, the method further comprising:
controlling movement of the window by the collimator to compensate for movement of the X-ray beam emitter between the first and second offset positions to provide alignment of the X-ray beam with the collector.

5. The method of claim 3, further comprising:
determining the first and second offset positions for the X-ray beam emitter based on levels of scatter of the X-ray beam detected in images obtained from the collector while the X-ray beam emitter is moved to each of the first and second offset positions.

6. The method of claim 3, further comprising:
angularly rotating the collector and the X-ray beam emitter toward each other in a first angular direction when the X-ray beam emitter and the collector are positioned with the first offset position, and to angularly rotate the collector and the X-ray beam emitter toward each other in a second angular direction opposite to the first angular direction when the X-ray beam emitter and the collector are positioned with the second offset position.

7. The method of claim 1, further comprising:
angularly rotating the X-ray beam emitter about an angular pivot between the first and second offset positions relative to the collector.

8. The method of claim 7, further comprising:
determining the first and second offset positions for the X-ray beam emitter based on levels of scatter of the X-ray beam detected in images obtained from the collector while the X-ray beam emitter is angularly rotated about the angular pivot between the first and second offset positions.

9. The method of claim 7, wherein the X-ray beam emitter includes a collimator that forms a window through which the X-ray beam is emitted toward the collector, the method further comprising:
controlling movement of the window by the collimator to compensate for rotation of the X-ray beam emitter about the angular pivot between the first and second offset positions to provide alignment of the X-ray beam with the collector.

10. The method of claim 1, wherein the collector is moveable along a linear rail, the method further comprising:
moving the collector along the linear rail between the first and second offset positions relative to the X-ray beam emitter.

11. A method for using a medical imaging system, said method comprising:
providing a medical imaging system including:
a movable station including a C-arm having a first end and a second end that are movable relative to the movable station,
a collector attached to the first end of the C-arm, and
a first X-ray beam emitter and a second X-ray beam emitter both facing the collector to emit X-ray beams in a direction of the collector and both attached to the second end of the C-arm;
moving one of the X-ray beam emitter and the collector to a first offset position relative to each other along a lateral axis;
obtaining a first set of images by moving the collector and the X-ray beam emitter about a scanned volume while the X-ray beam emitter and the collector are positioned with the first offset position;
moving the one of the X-ray beam emitter and the collector to a second offset position relative to each other along the lateral axis;
obtaining a second set of images by rotating the collector and the X-ray beam emitter about the scanned volume while the X-ray beam emitter and the collector are positioned with the second offset position; and
combining the first and second set of images to generate a three-dimensional image of the scanned volume

12. The method of claim 11, further comprising:
aligning a first edge of the X-ray beam with a first edge of the collector while the X-ray beam emitter and the collector are positioned with the first offset position; and
aligning a second edge of the X-ray beam, which is opposite to the first edge of the X-ray beam, with a second edge of the collector, which is opposite to the first edge of the collector, while the X-ray beam emitter and the collector are positioned with the second offset position.

13. The method of claim 11, further comprising:
moving the X-ray beam emitter along the linear rail between the first and second offset positions relative to the collector.

14. The method of claim 13, wherein the X-ray beam emitter comprises a collimator that forms a window through which the X-ray beam is emitted toward the collector, the method further comprising:
controlling movement of the window by the collimator to compensate for movement of the X-ray beam emitter along the linear rail between the first and second offset positions to provide alignment of the X-ray beam with the collector.

15. The method of claim 13, further comprising:
determining the first and second offset positions for the X-ray beam emitter based on levels of scatter of the X-ray beam detected in images obtained from the collector while the X-ray beam emitter is moved to each of the first and second offset positions.

16. The method of claim 13, further comprising:
angularly rotating the collector and the X-ray beam emitter toward each other in a first angular direction when the X-ray beam emitter and the collector are positioned with the first offset position, and to angularly rotate the collector and the X-ray beam emitter toward each other in a second angular direction opposite to the first angular direction when the X-ray beam emitter and the collector are positioned with the second offset position.

17. The method of claim 11, further comprising:
angularly rotating the X-ray beam emitter about an angular pivot between the first and second offset positions relative to the collector.

18. The method of claim 17, further comprising:
determining the first and second offset positions for the X-ray beam emitter based on levels of scatter of the X-ray beam detected in images obtained from the collector while the X-ray beam emitter is angularly rotated about the angular pivot between the first and second offset positions.

19. The method of claim 17, wherein the X-ray beam emitter includes a collimator that forms a window through which the X-ray beam is emitted toward the collector, the method further comprising:
controlling movement of the window by the collimator to compensate for rotation of the X-ray beam emitter about the angular pivot between the first and second offset positions to provide alignment of the X-ray beam with the collector.

20. The method of claim 11, wherein the collector is moveable along a linear rail, the method further comprising:
moving the collector along the linear rail between the first and second offset positions relative to the X-ray beam emitter.
